(19) United States
(12) Patent Application Publication
(10) Pub. No.: US 2017/0100317 A1
(43) Pub. Date: Apr. 13, 2017

(54) COMPOSITION COMPRISING A PARTICULAR PHENOLIC COUPLER AND AN OXIDATION BASE

(71) Applicant: L'OREAL, Paris (FR)

(72) Inventors: Alain LAGRANGE, Coupvray (FR); Boris LALLEMAN, Paris (FR)

(21) Appl. No.: 15/311,266

(22) PCT Filed: May 13, 2015
(86) PCT No.: PCT/EP2015/060637

§ 371 Date: Nov. 15, 2016

(30) Foreign Application Priority Data
May 16, 2014 (FR) 1454403

(51) Int. Cl. A61K 8/49; A61K 8/368; A61Q 5/10

(52) U.S. Cl. CPC A61K 8/494; A61Q 5/10; A61K 8/368; A61K 2800/4324; A61K 2800/882

(57) ABSTRACT

The invention relates to a composition comprising at least i) one coupler of formula (I), and also addition salts thereof, optical or geometrical isomers thereof, tautomers thereof or solvates thereof: in which formula (I): X represents a halogen atom, a COOH group or an amido group —C(O)—NR₅R₆ with R₅ and R₆, which may be identical or different, representing a hydrogen atom or an alkyl group, R₁ to R₄, which may be identical or different, represent a hydrogen or halogen atom, an alkyl group or an alkoxy group, and ii) at least one oxidation base. The invention also relates to a process for dyeing keratin fibres using said composition.

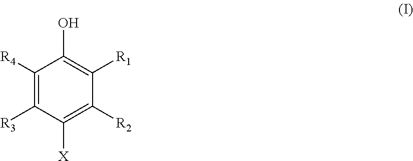

The invention relates to a composition comprising i) at least one particular coupler of para-substituted phenol type and ii) at least one oxidation base, and also to a process for dyeing keratin fibres using said composition.

Many people have sought for a long time to modify the colour of their hair and in particular to mask their grey hair.

It is known practice to dye keratin fibres, in particular human keratin fibres such as the hair, to obtain “permanent” colourings with dye compositions containing oxidation dye precursors, which are generally known as oxidation bases, such as ortho- or para-phenylenediamines, ortho- or para-aminophenols, or heterocyclic compounds such as pyrazoles, pyrazolinones or pyrazolo-pyridines. These oxidation bases are colourless or weakly coloured compounds, which, when combined with oxidizing products, may give rise to coloured compounds via a process of oxidative condensation.

It is also known that the shades obtained with these oxidation bases may be varied by combining them with couplers or colouring modifiers, the latter being chosen especially from aromatic meta-diamines, meta-aminophenols, meta-diphenols and certain heterocyclic compounds such as indole or pyridine compounds. The variety of molecules used as oxidation bases and couplers allows a wide range of colours to be obtained.

The oxidation dyeing process thus consists in applying to keratin fibres a dye composition comprising oxidation bases or a mixture of oxidation bases and couplers with hydrogen peroxide as oxidizing agent, in leaving it to diffuse, and then in rinsing the fibres.

However, the use of these dye compositions may have a certain number of drawbacks. Specifically, after application to keratin fibres, the dyeing power obtained may not be entirely satisfactory, or may even be weak, and lead to a restricted range of colours. The colourings may also not be sufficiently persistent with respect to external agents such as light, shampoo or perspiration, and may also be too selective, i.e. the difference in colouring is too great along the same keratin fibre that is differently sensitized between its end and its root.

It is also sought to use compositions that show good tolerance with respect to keratin materials, for example compounds of natural origin.

Document FR 1 060 793 discloses resorcinol derivatives used as oxidation dye precursors, but these compounds cannot satisfy the majority of the conditions required above.

One of the objects of the present invention is thus to propose compositions for dyeing keratin fibres, in particular human keratin fibres such as the hair, which are especially capable of leading to a wide range of colours with powerful, sparingly selective colourings that are resistant to external agents (such as shampoo, light, perspiration or bad weather), and which are well tolerated and friendly to keratin materials.

This aim is achieved by the present invention, one subject of which is especially a composition for dyeing keratin fibres, in particular human keratin fibres such as the hair, comprising:
i) at least one coupler of formula (I), and also organic or mineral acid or base salts thereof, optical or geometrical isomers thereof, tautomers thereof or solvates thereof: in which formula (I):

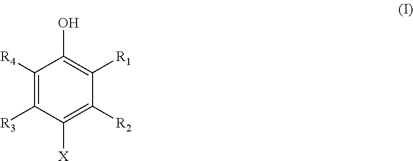

X represents a halogen atom, a COOH group or an amido group
—C(O)—NR₅R₆ with R₅ and R₆, which may be identical or different, representing a hydrogen atom or an alkyl group,
R₁ to R₄, which may be identical or different, represent a hydrogen or halogen atom, an alkyl group or an alkoxy group,
and
ii) at least one oxidation base.

A subject of the invention is also a process for dyeing keratin fibres using this composition.

Another subject of the invention is the use of the compounds of formula (I) for the oxidation dyeing of keratin fibres, in particular human keratin fibres such as the hair.

These compounds, some of which are of natural origin, make it possible to obtain compositions that are comfortable to use and well tolerated by keratin materials.

The composition of the invention leads to particularly powerful, chromatic and sparingly selective colourings. The colour build-up on keratin fibres treated with the composition of the invention is very satisfactory. It also makes it possible to obtain colourings that are resistant to the various attacking factors to which the hair may be subjected, such as light, bad weather and perspiration. The compositions in accordance with the invention also make it possible to achieve shades in a very broad range of colours.

For the purposes of the present invention, the term “build-up” of the colour of the keratin fibres means the variation in colouring between locks of non-dyed grey hair and locks of dyed hair.

Other subjects, characteristics, aspects and advantages of the present invention will emerge even more clearly on reading the description and the examples that follow.

In the text hereinbelow, and unless otherwise indicated, the limits of a range of values are included within that range.

The expression “at least one” is equivalent to the expression “one or more”.
an “alkyl” radical is a linear or branched C₁-C₂₀, better still C₁-C₁₀, preferably C₁-C₆ and particularly C₁-C₄ hydrocarbon-based radical, such as methyl or ethyl;
the term “optionally substituted” attributed to the alkyl radical means that said alkyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom, v) phenyl, vi) (C₁-C₆)alkoxycarbonyl, vii) (C₁-C₆)alkylcarbonyloxy, viii) H—C(O)—O—;
an “alkoxy” radical is an alkyl-oxy or alkyl-O— radical for which the alkyl radical is a linear or branched C₁-C₁₆ and preferentially C₁-C₈ hydrocarbon-based radical; particularly C₁-C₄ such as methoxy or ethoxy, and when the alkoxy group is optionally substituted, this means that the alkyl group is optionally substituted as defined above;
an anionic counterion is organic or mineral, preferentially chosen from halide anions such as Cl⁻, Br⁻ or I⁻, and organic anions such as mesylates.

In general, the term “addition salts” of compounds means the addition salts of these compounds either with an organic or mineral acid, such as hydrochlorides, hydrobromides, sulfates, citrates, succinates, tartrates, lactates, tosylates, benzenesulfonates, dodecylbenzenesulfonates, phosphates and acetates, and preferably the hydrochlorides, citrates, succinates, tartrates, phosphates and lactates, or with an organic or mineral base, such as alkali metal or alkaline-earth metal hydroxides or carbonates, ammonia, or organic amines such as alkanolamines.

The solvates of compounds more particularly represent the hydrates of such compounds and/or the combination of such compounds with a linear or branched C₁-C₄ alcohol such as methanol, ethanol, isopropanol or n-propanol. Preferably, the solvates are hydrates.

The composition of the present invention is particularly useful for the oxidation dyeing of keratin fibres, in particular of human keratin fibres.

i) Coupler of Formula (I)

The composition according to the invention comprises at least i) one coupler of formula (I), and also addition salts thereof, optical or geometrical isomers thereof, tautomers thereof or solvates thereof:

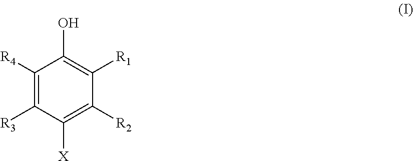

in which formula (I):
X represents a halogen atom, a COOH group or an amido group
—C(O)—NR₅R₆ with R₅ and R₆, which may be identical or different, representing a hydrogen atom or an alkyl group,
R₁ to R₄, which may be identical or different, represent a hydrogen or halogen atom, an alkyl group or an alkoxy group,

and

ii) at least one oxidation base.
Preferably, X represents a halogen atom, preferably bromine or chlorine, or a COOH group.
The alkyl or alkoxy groups may be linear or branched. They may be optionally substituted, and preferably the alkyl or alkoxy groups are unsubstituted. More preferably, the alkyl or alkoxy groups are unsubstituted C₁-C₂₀ groups.
Preferably, in formula (I), R₁ to R₄, which may be identical or different, represent a hydrogen or halogen atom; a C₁-C₁₀, preferably C₁-C₈ and better still C₁-C₆ alkyl group, preferably a methyl group, or a C₁-C₁₀, preferably C₁-C₈ and better still C₁-C₆ alkoxy group, preferably a methoxy group.
According to a preferred embodiment, the compound of formula (I) is such that R₂ and R₃ represent a hydrogen atom.
According to a preferred embodiment, R₁ and R₄, which may be identical or different, independently represent a hydrogen or halogen atom, a C₁-C₆ alkyl group, preferably a methyl group, or a C₁-C₆ alkoxy group, preferably a methoxy group.
More preferably, the compound of formula (I) is such that R₁ represents a hydrogen atom, a C₁-C₆ alkyl group, preferably a methyl group, or a C₁-C₆ alkoxy group, preferably a methoxy group.
More preferably, R₄ represents a hydrogen or halogen atom, preferably bromine or chlorine, a C₁-C₆ alkyl group, preferably a methyl group, or a C₁-C₆ alkoxy group, preferably a methoxy group.
According to a specific embodiment, the compound of formula (I) is such that:
X represents a COOH group,
R₂ and R₃ represent a hydrogen atom and
R₁ and R4 represent a C₁-C₆ alkyl group, preferably a methyl group.
The compounds of formula (I) may be chosen in particular from the following compounds:

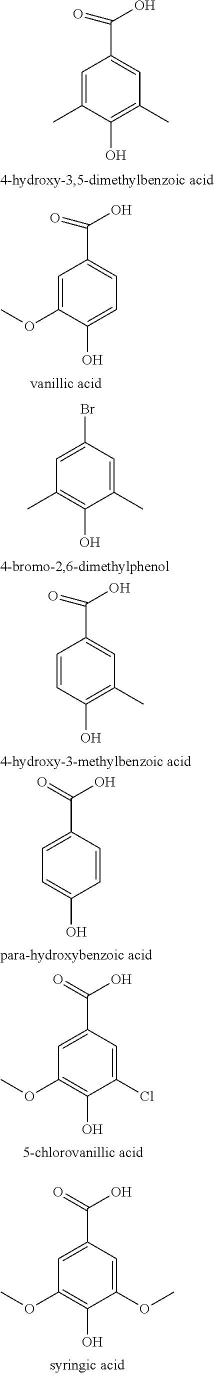

and mixtures thereof.

According to a specific embodiment, the compound of formula (I) is 4-hydroxy-3,5-dimethylbenzoic acid.

The compound(s) of formula (I) may be present in the composition of the invention in an amount ranging from 0.0001% to 20% by weight approximately, preferably from 0.005% to 10% and better still from 0.01% to 6% by weight relative to the total weight of the composition.

### ii) Oxidation Base

The composition of the invention comprises one or more oxidation bases ii) other than the compound of formula (I).

In particular, the oxidation bases are chosen from para-phenylenediamines, bis(phenyl)alkylenediamines, para-aminophenols, ortho-aminophenols and heterocyclic bases, and the addition salts thereof. Preferentially, the oxidation base(s) of the invention are chosen from para-phenylenediamines and heterocyclic bases.

Among the para-phenylenediamines, examples that may be mentioned include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N, N-dipropyl-para-phenylenediamine, 4-amino-N,N-diethyl-3-methylaniline, N,N-bis(β-hydroxyethyl)-para-phenylenediamine, 4-N,N-bis(β-hydroxyethypamino-2-methylaniline, 4-N,N-bis(β-hydroxyethypamino-2-chloroaniline, 2-β-hydroxyethyl-para-phenylenediamine, 2-fluoro-para-phenylenediamine, 2-isopropyl-para-phenylenediamine, N-(β-hydroxypropyly para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-methoxymethyl-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine and 2-methoxymethyl-para-phenylenediamine, and the addition salts thereof with an acid, are particularly preferred.

Among the bis(phenyl)alkylenediamines, examples that may be mentioned include N,N′-bis(β-hydroxyethyl)-N,N═-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and the addition salts thereof.

Among the para-aminophenols, examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols, examples that may be mentioned include 2-aminophenol, 2-amino-5-methylphenol, 2-amino-6-methylphenol, 5-acetamido-2-aminophenol and the addition salts thereof.

Among the heterocyclic bases, examples that may be mentioned include pyridine derivatives, pyrimidine derivatives and pyrazole derivatives.

According to a preferred embodiment, the oxidation bases ii) are chosen from heterocyclic bases, in particular pyridine derivatives, pyrimidine derivatives and pyrazole derivatives, and mixtures thereof.

More preferably, the oxidation bases ii) are chosen from the compounds of formula (II), addition salts thereof and solvates thereof:

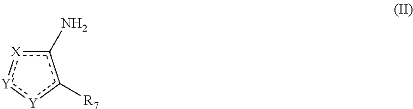

in which formula (I):
X denotes a group —CO— or —CR₁₀—,
Y denotes a nitrogen atom or a group —NR₈,
Y′ denotes a nitrogen atom or a group —NR₉,
with R₁₀ representing a hydrogen atom or a C₁-C₄ alkyl group, optionally substituted with one or more hydroxyl or amino groups,
R₈ representing a C₁-C₄ alkyl group substituted with one or more hydroxyl or amino groups,
R₉ representing a C₁-C₄ alkyl group optionally substituted with one or more hydroxyl or amino groups,
it being possible for R₈ and R₉, on the one hand, and for R₉ and R₁₀, on the other hand, to form, with the atoms bearing them, a saturated or unsaturated, 5- to 7-membered heterocycle, optionally substituted with one or more halogen atoms or one or more hydroxyl, amino or C₁-C₄ alkyl groups,
R₇ representing an amino group, a C₁-C₄ alkyl group optionally substituted with one or more hydroxyl or amino groups, or a C₁-C₄ alkoxy group optionally substituted with one or more hydroxyl or amino groups,
the ring containing X, Y and Y′ comprising at least one double bond.

The oxidation bases of formula (II) or the addition salts thereof according to the invention may be present in the form of solvates, for example hydrates or solvates of a linear or branched alcohol such as ethanol or isopropanol.

In a first preferred variant of the invention, the oxidation base(s) are chosen from the compounds of formula (II'), addition salts thereof and solvates thereof:

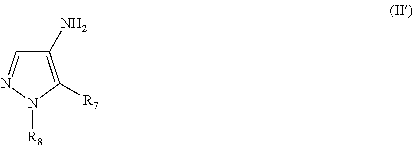

R₇ and R₈ having the same definitions as previously.

Preferably, R₈ represents a C₁-C₄ alkyl group substituted with at least one hydroxyl group, in particular a hydroxyethyl group, and R₇ represents an amino group.

In this variant, preference will be given to using 2-(4,5-diamino-1H-pyrazol-1-yl)ethanol or an addition salt thereof.

In a second preferred variant of the invention, R₈ and R₉, on the one hand, or R₉ and R₁₀, on the other hand, form, with the atoms bearing them, a saturated or unsaturated, 5- to 7-membered heterocycle optionally substituted with one or more halogen atoms, or one or more hydroxyl, amino and/or C₁-C₄ alkyl groups.

In this second preferred variant, the oxidation base(s) are preferably chosen from:
a) the compounds of formula (II″), addition salts thereof and solvates thereof:

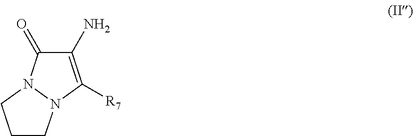

R₇ having the same meaning as before, and preferably R₇ representing an amino group;
b) the compounds of formula (II″), addition salts thereof and solvates thereof:

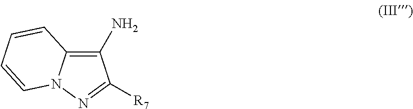

R₇ having the same meaning as previously, R₇ preferably representing a C₁-C₄ alkoxy group optionally substituted with one or more hydroxyl groups, in particular a hydroxyethoxy group; and
c) mixtures thereof.
   Among these compounds of formula (II″), preference will be given to using 2,3-diamino-6,7-dihydro-1H,5H-pyrazolo[1,2-a]pyrazol-1-one or an addition salt thereof.
   Among these compounds of formula (II″'), preference will be given to using 2-{[3-aminopyrazolo[1,5-a]pyrid-2-yl]oxy}ethanol or an addition salt thereof.

Mention may be made also of 3-aminopyrazolo-[1,5-a]-pyridines substituted on carbon atom 2 by one heterocycloalkyl group containing from 5 to 7 members chain, and from 1 to 3 heteroatomes, optionally cationic, optionally substituted by one or more (C₁-C₆)alkyl group, such as di(C₁-C₄)alkylpiperazinium, such as 4-(3-aminopyrazolo[1,5-a]pyridin-2-yl)-1,1-dimethylpiperazin-1-ium, especially 4-(3-aminopyrazolo[1,5-a]pyridin-2-yl)-1,1-dimethylpiperazin-1-ium chloride hydrochloride, or one of their addition salt and mixture thereof.

The heterocyclic oxidation bases are preferably chosen from 2-(4,5-diamino-1H-pyrazol-1-yl)éthanol, 2,3-diamino-6,7-dihydro-1H,5H-pyrazolo[1,2-A]pyrazol-1-one, 2-[3-aminopyrazolo[1,5-a]pyridin-2-yl]oxy]éthanol, 4-(3-aminopyrazolo[1,5-a]pyridin-2-yl)-1,1-dimethylpiperazin-1-ium or one of their addition salt and mixture thereof.

According to a specific embodiment, the oxidation bases ii) are chosen from paraphenylenediamines and bis(phenyl)alkylene diamines, in particular those previously mentioned and more particularly from N,N-bis-(β-hydroxyethyl) paraphénylènediamine and salts thereof.

Preferably in this embodiment, the compound of formula (I) is 4-hydroxy-3,5-dimethylbenzoic acid.

According to an other specific embodiment, the oxidation bases ii) are chosen from heterocyclic oxidation bases, in particular those previously mentioned and more preferably chosen from 2-(4,5-diamino-1H-pyrazol-1-yl)éthanol, 2,3-diamino-6,7-dihydro-1H,5H-pyrazolo[1,2-A]pyrazol-1-one, 2-[3-aminopyrazolo[1,5-a]pyridin-2-yl]oxy]éthanol, 4-(3-aminopyrazolo[1,5-a]pyridin-2-yl)-1,1-dimethylpiperazin-1-ium or one of their addition salt and mixture thereof he oxidation base(s) present in the composition of the invention are generally present in an amount ranging from 0.0001% to 20% by weight approximately, preferably ranging from 0.005% to 10% and better still from 0.01% to 6% by weight, relative to the total weight of the composition.

### Additional Couplers:

The composition according to the invention may also contain, besides the coupler of formula (I) defined above, one or more additional couplers conventionally used for the dyeing of keratin fibres. Among these couplers, mention may be made especially of meta-phenylenediamines, meta-aminophenols, meta-diphenols, naphthalene-based couplers and heterocyclic couplers, and also the addition salts thereof.

Examples that may be mentioned include 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole, 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the addition salts thereof with an acid, and mixtures thereof.

In general, the additional addition salts of the oxidation bases and couplers that may be used in the context of the invention are especially chosen from the addition salts with an acid such as the hydrochlorides, hydrobromides, sulfates, citrates, succinates, tartrates, lactates, tosylates, benzenesulfonates, phosphates and acetates.

In the composition of the present invention, the additional coupler(s) are generally present in an amount ranging from 0.001% to 20% by weight approximately and preferably ranging from 0.005% to 6% by weight relative to the total weight of the dye composition.

### Additional Dyes:

According to a particular embodiment of the invention, the composition also comprises one or more synthetic or natural direct dyes, chosen from ionic or nonionic species, preferably cationic or nonionic species.

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanine dyes, and natural direct dyes, alone or as mixtures.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions comprising these natural dyes and in particular henna-based extracts or poultices.

The direct dye(s), if they are present, more particularly represent from 0.001% to 10% by weight relative to the total weight of the composition, and preferably from 0.005% to 6% by weight relative to the total weight of the composition.

iii) Fatty Substances:

According to a preferred embodiment of the invention, the composition of the invention comprises one or more fatty substances, preferably one or more fatty substances that are liquid at room temperature and atmospheric pressure (or oil).

The term “fatty substance” means an organic compound that is insoluble in water at ordinary room temperature (25° C.) and at atmospheric pressure (760 mmHg) (solubility of less than 5%, preferably less than 1% and even more preferentially less than 0.1%). They bear in their structure at least one hydrocarbon-based chain comprising at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

These fatty substances are neither polyoxyethylenated nor polyglycerolated. They are different from fatty acids since salified fatty acids constitute soaps which are generally soluble in aqueous media.

The fatty substances are especially chosen from C₆-C₁₆ hydrocarbons or hydrocarbons comprising more than 16 carbon atoms and in particular alkanes, oils of animal origin, oils of plant origin, synthetic glycerides, natural triglycerides, fluoro oils of synthetic origin, fatty alcohols other than natural or synthetic glycerides, fatty acid and/or fatty alcohol esters, non-silicone waxes, and silicones.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, dodecane, undecane, tridecane, and isoparaffins, for instance isohexadecane and isodecane. The linear or branched hydrocarbons containing more than 16 carbon atoms may be chosen from liquid paraffins, petroleum jelly, liquid petroleum jelly, polydecenes, and hydrogenated polyisobutene such as Parleam®.

Among the animal oils, mention may be made of perhydrosqualene. Among the triglycerides of plant or synthetic origin, mention may be made of liquid fatty acid triglycerides comprising from 6 to 30 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, castor oil, avocado oil, jojoba oil, shea butter oil, caprylic/capric acid triglycerides, for instance those sold by the company Stéarineries Dubois or those sold under the names Miglyol® 810, 812 and 818 by the company Dynamit Nobel.

Among the fluoro oils, mention may be made of perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The liquid fatty alcohols that may be used in the cosmetic compositions of the invention are saturated or unsaturated, and linear or branched, and comprise from 6 to 30 carbon atoms and more particularly from 8 to 30 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

The wax(es) that may be used in the cosmetic composition are chosen especially from carnauba wax, candelilla wax, esparto grass wax, paraffin wax, ozokerite, plant waxes, for instance olive wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

As regards the esters of fatty acids and/or of fatty alcohols, other than the triglycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters more particularly being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as fatty ester, sugar esters and diesters of C₆-C₃₀ and preferably C₁₂-C₂₂ fatty acids. It is recalled that the term “sugar” is intended to mean oxygen-bearing hydrocarbon-based compounds bearing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, in particular alkyl derivatives, such as methyl derivatives, for instance methylglucose.

The sugar and fatty acid esters may be chosen in particular from the group comprising the esters or mixtures of sugar esters described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates or arachidonates, or mixtures thereof such as, in particular, oleate/palmitate, oleate/stearate or palmitate/stearate mixed esters.

More particularly, use is made of monoesters and diesters and in particular mono- or di-oleate, -stearate, -behenate, -oleate/palmitate, -linoleate, -linolenate or -oleate/stearate of sucrose, glucose or methylglucose.

An example that may be mentioned is the product sold under the name Glucate® DO by the company Amerchol, which is a methylglucose dioleate.

Examples of esters or mixtures of esters of sugar of fatty acid that may also be mentioned include:

the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;

the products sold under the name Ryoto Sugar Esters, for example referenced B370 and corresponding to sucrose behenate formed from 20% monoester and 80% di-triester-polyester;

the sucrose mono-di-palmitostearate sold by the company Goldschmidt under the name Tegosoft® PSE.

The silicones that may be used in the cosmetic composition (A) according to the present invention are volatile or non-volatile, cyclic, linear or branched silicones, which are unmodified or modified with organic groups, having a viscosity from 5×10⁻⁶ to 2.5 m²/s at 25° C., and preferably 1×10⁻⁵ to 1 m²/s.

The silicones that may be used in accordance with the invention may be in the form of oils, waxes, resins or gums.

Preferably, the silicone is chosen from polydialkylsiloxanes, in particular polydimethylsiloxanes (PDMSs), and organomodified polysiloxanes comprising at least one functional group chosen from poly(oxyalkylene) groups, amino groups and alkoxy groups.

Organopolysiloxanes are defined in greater detail in Walter Noll's Chemistry and Technology of Silicones (1968), Academic Press. They may be volatile or non-volatile.

When they are volatile, the silicones are more particularly chosen from those with a boiling point of between 60° C. and 260° C., and even more particularly from:

cyclic polydialkylsiloxanes containing from 3 to 7 and preferably from 4 to 5 silicon atoms. These are, for example, octamethylcyclotetrasiloxane sold in particular under the name Volatile Silicone® 7207 by Union Carbide or Silbione® 70045 V 2 by Rhodia, decamethylcyclopentasiloxane sold under the name Volatile Silicone® 7158 by Union Carbide, and Silbione® 70045 V 5 by Rhodia, and mixtures thereof.

Mention may also be made of cyclocopolymers of the dimethylsiloxanes/methylalkylsiloxane type, such as Volatile Silicone® FZ 3109 sold by the company Union Carbide, of formula:

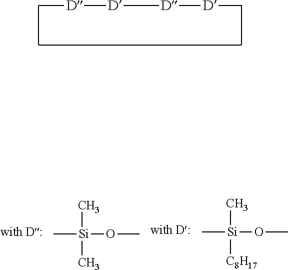

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10-⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics.

Use is preferably made of non-volatile polydialkylsiloxanes, polydialkylsiloxane gums and resins, polyorganosiloxanes modified with the organofunctional groups above, and mixtures thereof.

These silicones are more particularly chosen from polydialkylsiloxanes, among which mention may be made mainly of polydimethylsiloxanes bearing trimethylsilyl end groups. The viscosity of the silicones is measured at 25° C. according to ASTM Standard 445 Appendix C.

Among these polydialkylsiloxanes, mention may be made, in a nonlimiting manner, of the following commercial products:

the Silbione® oils of the 47 and 70 047 series or the Mirasil® oils sold by Rhodia, for instance the oil 70 047 V 500 000;

the oils of the Mirasil® series sold by the company Rhodia;

the oils of the 200 series from the company Dow Corning, such as DC200 with a viscosity of 60 000 mm²/s;

the Viscasil® oils from General Electric and certain oils of the SF series (SF 96, SF 18) from General Electric.

Mention may also be made of polydimethylsiloxanes bearing dimethylsilanol end groups known under the name dimethiconol (CTFA), such as the oils of the 48 series from the company Rhodia.

In this category of polydialkylsiloxanes, mention may also be made of the products sold under the names Abil Wax® 9800 and 9801 by the company Goldschmidt, which are poly(C₁-C₂₀)dialkylsiloxanes.

The silicone gums that may be used in accordance with the invention are especially polydialkylsiloxanes and preferably polydimethylsiloxanes with high number-average molecular weights of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Products that may be used more particularly in accordance with the invention are mixtures such as:

the mixtures formed from a hydroxy-terminated polydimethylsiloxane or dimethiconol (CTFA) chain, and from a cyclic polydimethylsiloxane, also known as cyclomethicone (CTFA), such as the product Q2 1401 sold by the company Dow Corning;

mixtures of a polydimethylsiloxane gum and a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs with different viscosities, and more particularly of a PDMS gum and of a PDMS oil, such as the product SF 1236 from the company General Electric. The product SF 1236 is a mixture of a gum SE 30 defined above with a viscosity of 20 m²/s and of an oil SF 96 with a viscosity of 5×10⁻⁶ m²/s. This product preferably comprises 15% of gum SE 30 and 85% of an oil SF 96.

The organopolysiloxane resins that may be used in accordance with the invention are crosslinked siloxane systems containing the following units:

R₂SiC_{2/2}, R₃SiC_{1/2}, RSiO_{3/2} and SiO_{4/2},

in which R represents an alkyl containing 1 to 16 carbon atoms. Among these products, the ones that are particularly preferred are those in which R denotes a C₁-C₄ lower alkyl group, more particularly methyl.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethylsiloxysilicate type resins sold in particular under the names X22-4914, X21-5034 and X21-5037 by the company Shin-Etsu.

The organomodified silicones that may be used in accordance with the invention are silicones as defined previously and comprising in their structure one or more organofunctional groups attached via a hydrocarbon-based group.

Besides the silicones described above, the organomodified silicones may be polydiarylsiloxanes, in particular polydiphenylsiloxanes, and polyalkylarylsiloxanes functionalized by the organofunctional groups mentioned previously.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, examples that may be mentioned include the products sold under the following names:

the Silbione® oils of the 70 641 series from Rhodia;

the oils of the Rhodorsil® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amino groups, such as the products sold under the names GP 4 Silicone Fluid and GP 7100 by the company Genesee or the products sold under the names Q2 8220 and Dow Corning 929 or 939 by the company Dow Corning. The substituted amine groups are, in particular, C₁-C₄ aminoalkyl groups;

alkoxy groups such as the product sold under the name Silicone Copolymer F-755 by SWS Silicones, and Abil Wax® 2428, 2434 and 2440 by the company Goldschmidt.

The fatty substances are advantageously chosen from C₆-C₁₆ hydrocarbons or hydrocarbons comprising more than 16 carbon atoms, and in particular alkanes, oils of plant origin, fatty alcohols, fatty acid and/or fatty alcohol esters other than triglycerides, and silicones, or mixtures thereof.

Preferably, the fatty substance is an oil (a compound that is liquid at a temperature of 25° C. and at atmospheric pressure).

Preferably, the fatty substance is chosen from liquid petroleum jelly, C₆-C₁₆ alkanes, polydecenes, liquid esters of a fatty acid and/or of a fatty alcohol, and liquid fatty alcohols, or mixtures thereof. Better still, the fatty substance is chosen from liquid petroleum jelly and octyldodecanol.

According to a preferred embodiment, the fatty substances, which are preferably liquid, are present in a content of greater than or equal to 10% by weight, relative to the total weight of the composition, preferably greater than or equal to 20%, better still greater than or equal to 30% by weight and even better still greater than or equal to 40% by weight.

The fatty substance content in the composition according to the invention may range from 5% to 90% by weight, preferably from 10% to 80% by weight, more particularly from 20% to 80% by weight, better still from 25% to 75% by weight and most particularly from 40% to 70% by weight relative to the total weight of the composition.

iv) Metal Catalysts

According to a particular embodiment of the invention, the composition according to the invention also comprises one or more metal catalysts.

“Metal catalysts” are compounds that comprise one or more metals in their structure.

The metals are chosen from transition metals and rare-earth metals, and alloys thereof. In particular, the metals are chosen from transition metals and rare-earth metals.

Among the transition metals, mention may be made especially of manganese, iron, cobalt, copper, zinc, platinum, nickel, titanium, silver, zirconium, chromium, molybdenum, tungsten, platinum, gold and vanadium, and among these most particularly manganese.

Among the rare-earth metals, mention may particularly be made of cerium.

Thus, the metal catalysts are especially catalysts based on transition metals or on rare-earth metals, and more particularly manganese-based, vanadium-based or cerium-based catalysts.

The metal catalysts used may be in the form of metal salts, metal oxides or metal complexes, and mixtures thereof.

For the purposes of the present invention, the term “metal complexes” means systems in which the metal ion, i.e. the central atom, is bonded to one or more electron donors, called ligands, via chemical bonds. Examples that may be mentioned include porphyrins and phthalocyanines, which are especially cationic.

Preferably, the metal catalysts used in the dyeing process are chosen from metal salts.

For the purposes of the present invention, the term “metal salts” means salts derived from the action of an acid on a metal.

Preferentially, the metal catalysts used in the dyeing process are chosen from transition metal salts, such as manganese salts, and rare-earth metal salts, such as cerium salts, and also mixtures thereof.

The metal salts may be mineral or organic salts.

According to one variant, the metal salts are mineral and may be chosen from halides, carbonates, sulfates and phosphates, in particular optionally hydrated halides.

According to another preferred variant, the metal salts are in oxidation state II and bear two ligands derived from a C₂-C₁₀ carboxylic acid or (poly)hydroxy acid.

The term “carboxylic acid” means a carboxylic acid comprising a hydrocarbon-based chain which is linear or branched, and saturated or unsaturated, preferably saturated and/or linear, comprising from 1 to 10 carbon atoms, and comprising from 1 to 4 carboxylic groups —C(O)—OH, at least one of said —O(O)—OH functions of which is in the carboxylate form —C(O)—O⁻ complexed with the metal atom, preferably Mn(II).

The term “(poly)hydroxy acid” means any carboxylic acid which comprises a hydrocarbon-based chain which is linear or branched, and saturated or unsaturated, preferably saturated and/or linear, comprising from 1 to 10 carbon atoms and from 1 to 9 hydroxyl groups, and comprising from 1 to 4 carboxylic groups —O(O)—OH, at least one of said —O(O)—OH functions of which is in the carboxylate form —O(O)—O⁻ complexed with the metal atom, preferably Mn(II).

More particularly, the metal salt is complexed with two carboxylate groups such as that of formula (II):

Rₐ—C(O)—O-M-O—C(O)—R_{b} (II)

and also the solvates thereof, such as hydrates, and enantiomers thereof, in which formula (II):
M represents a metal (II) or metal²⁺ in oxidation state 2,
Rₐ and R_{b}, which may be identical or different, represent a (poly)(hydroxy)(C₁-C₆)alkyl group. The metal catalysts are particularly chosen from organic acid salts of transition metals, especially of manganese, and mineral salts of rare-earth metals, especially of cerium.

According to one particular embodiment of the invention, the manganese is not a manganese oxide, but a manganese salt.

The organic metal salts may be more particularly chosen from organic acid salts such as citrates, lactates, glycolates, gluconates, acetates, propionates, fumarates, oxalates and tartrates, especially gluconates.

More preferentially, the metal catalysts are chosen from manganese gluconate and cerium chloride heptahydrate, in particular manganese gluconate. Preferably, the metal catalyst(s) are chosen from the compounds of formula (II) and more particularly represent manganese gluconate.

The metal catalysts may be present in a content ranging from 0.001% to 10% by weight, preferably in a content ranging from 0.001% to 1% by weight, better still ranging from 0.01% to 0.5% by weight relative to the total weight of the composition.

The composition of the invention is preferably cosmetic. The composition according to the invention may be an aqueous composition.

The term “aqueous composition” means a composition comprising more than 5% by weight of water, preferably more than 10% by weight of water and even more advantageously more than 20% by weight of water.

Even more preferentially, the water concentration of the compositions of the invention may range from 10% to 90% and better still from 20% to 80% of the total weight of the composition. The compositions of the invention may optionally comprise one or more organic solvents.

Examples of organic solvents that may be mentioned include linear or branched C₂-C₄ alkanols, such as ethanol and isopropanol; polyols and polyol ethers, for instance 2-butoxyethanol, glycerol, propylene glycol, dipropylene glycol, propylene glycol monomethyl ether, diethylene glycol monomethyl ether and monoethyl ether, and also aromatic alcohols, for instance benzyl alcohol or phenoxyethanol, and mixtures thereof.

The organic solvents, if they are present, represent a content usually ranging from 1% to 40% by weight and preferably ranging from 5% to 30% by weight relative to the weight of the composition containing them.

### The Adjuvants:

The composition of the invention comprising the coupler(s) of formula (I) of the invention may also contain additional ingredients.

The composition in accordance with the invention may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, film-forming agents, ceramides, preserving agents and opacifiers.

The above adjuvants are generally present in an amount, for each of them, of between 0.01% and 20% by weight, relative to the weight of the composition.

Needless to say, a person skilled in the art will take care to select this or these optional additional compound(s) such that the advantageous properties intrinsically associated with the composition in accordance with the invention are not, or are not substantially, adversely affected by the envisaged addition(s).

The composition according to the invention may be in various forms, such as in the form of liquids, creams or gels, or in any other form that is suitable for dyeing keratin fibres, and in particular human hair.

### The pH

The pH of the composition in accordance with the invention generally ranges from 2 to 12 and preferably from 4 to 11. It may be adjusted to the desired value by means of additional acidifying or basifying agents, such as those mentioned below usually used in the dyeing of keratin fibres, or alternatively using standard buffer systems.

Among the acidifying agents, examples that may be mentioned include mineral or organic acids, for instance hydrochloric acid, orthophosphoric acid, sulfuric acid, carboxylic acids, for instance acetic acid, tartaric acid, citric acid and lactic acid, and sulfonic acids.

### v) The Alkaline Agents:

Preferably, the dye composition comprises one or more organic or mineral, preferably organic, alkaline agents.

The alkaline agent(s) may be mineral or organic.

The mineral alkaline agent(s) are preferably chosen from aqueous ammonia, alkali metal carbonates or bicarbonates such as sodium or potassium carbonates and sodium or potassium bicarbonates, sodium hydroxide or potassium hydroxide, or mixtures thereof.

The organic alkaline agent(s) are preferably chosen from organic amines with a pK_{b} at 25° C. of less than 12, preferably less than 10 and even more advantageously less than 6. It should be noted that it concerns the pK_{b} corresponding to the functional group having the highest basicity. In addition, the organic amines do not comprise any alkyl or alkenyl fatty chain comprising more than ten carbon atoms.

The organic alkaline agent(s) are chosen, for example, from alkanolamines, oxyethylenated and/or oxypropylenated ethylenediamines, amino acids and the compounds of formula (Ill) below:

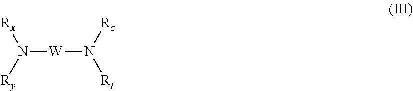

in which formula (III) W is a divalent C₁-C₆ alkylene radical optionally substituted with one or more hydroxyl groups or a C₁-C₆ alkyl radical, and/or optionally interrupted with one or more heteroatoms such as O, or NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (III) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Organic amines chosen from alkanolamines such as monoalkanolamines, dialkanolamines or trialkanolamines comprising one to three identical or different C₁-C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that can be used are of natural or synthetic origin, in their L, D or racemic form, and comprise at least one acid function chosen more particularly from carboxylic acid, sulfonic acid, phosphonic acid and phosphoric acid functions. The amino acids may be in neutral or ionic form.

As amino acids that may be used in the present invention, mention may be made especially of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (IV) below, and also the salts thereof R—CH₂—CH(NH₂)—C(O)—OH (IV) in which R represents a group chosen from imidazolyl, preferably imidazolyl-4-yl; aminopropyl; aminoethyl; —(CH₂)₂N(H)—C(O)—NH₂; and —(CH₂)₂—N(H)—C(NH)—NH₂.

The compounds corresponding to formula (IV) are histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that may be used in the present invention, mention may be made especially of carnosine, anserine and baleine.

The organic amine may also be chosen from compounds comprising a guanidine function. As amines of this type that may be used in the present invention, besides arginine, which has already been mentioned as an amino acid, mention may be made especially of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

Hybrid compounds that may be mentioned include the salts of the amines mentioned previously with acids such as carbonic acid or hydrochloric acid.

Guanidine carbonate or monoethanolamine hydrochloride may be used in particular.

Preferably, the alkaline agent(s) present in the composition of the invention are chosen from aqueous ammonia, alkanolamines, amino acids in neutral or ionic form, in particular basic amino acids, and preferably corresponding to those of formula (III).

Even more preferentially, the alkaline agent(s) are chosen from aqueous ammonia and alkanolamines, most particularly monoethanolamine (MEA).

Better still, the alkaline agent(s) are chosen from alkanolamines, most particularly monoethanolamine (MEA).

vi) Chemical Oxidizing Agent:

According to a particular embodiment of the invention, the composition according to the invention comprises at least one chemical oxidizing agent. The term “chemical oxidizing agent” means an oxidizing agent other than atmospheric oxygen.

The oxidizing agents conventionally used for the oxidation dyeing of keratin fibres are, for example, hydrogen peroxide, urea peroxide, alkali metal bromates, persalts such as perborates and persulfates, peracids and oxidase enzymes, among which mention may be made of peroxidases. Hydrogen peroxide is particularly preferred. More particularly, the chemical oxidizing agent(s) are chosen from hydrogen peroxide, urea peroxide, alkali metal bromates, peroxygenated salts, for instance persulfates or perborates, peracids and precursors thereof and alkali metal or alkaline-earth metal percarbonates. The chemical oxidizing agent is advantageously hydrogen peroxide. The concentration of chemical oxidizing agents may range more particularly from 0.1% to 50% by weight, even more preferentially from 0.5% to 20% by weight and better still from 1% to 15% by weight relative to the weight of the composition comprising it.

According to one embodiment, the chemical oxidizing agent(s) are present in an “oxidizing” composition to be used and preferably to be mixed at the time of use with the composition comprising one or more compounds of formula (I).

The oxidizing composition may also contain various adjuvants conventionally used in compositions for dyeing the hair and as defined previously.

### Dyeing Process:

Another subject of the invention concerns a process for dyeing keratin fibres, in particular human keratin fibres such as the hair, which consists in using the composition according to the invention comprising at least compounds i) and ii) as defined previously.

In particular, the process of the present invention is a process in which a dye composition according to the invention as defined previously is applied to the keratin fibres, in the presence of a chemical oxidizing agent as defined previously, for a time that is sufficient to develop the desired colouring.

The colour may be revealed at acidic, neutral or alkaline pH, and the chemical oxidizing agent(s) may be added to the dye composition of the invention just at the time of use, or they may be used starting with an oxidizing composition containing them, which is applied simultaneously with or sequentially to the composition of the invention. According to one embodiment, the oxidizing composition comprises at least one fatty substance, which is preferably liquid, chosen from the fatty substances mentioned above. More particularly, the oxidizing composition has a content of fatty substance, which is preferably liquid, of greater than or equal to 10% by weight, preferably greater than or equal to 15% by weight and better still greater than or equal to 20% by weight relative to the total weight of the composition.

The content of fatty substance in the oxidizing composition may range from 10% to 90% by weight, even more particularly ranging from 20% to 80% by weight and better still from 20% to 50% by weight of the oxidizing composition.

According to one embodiment of the invention, the composition which comprises the ingredients i) and ii) and optionally iii) to vi) as described previously is obtained by mixing at least two compositions, preferably three or four compositions.

According to one particular variant of the invention, the process uses a dye composition which is derived from mixing two compositions:

a composition (A) comprising:
at least i) one oxidation base as defined previously;
at least ii) one coupler of formula (I) as defined previously;
optionally at least one alkaline agent v) as defined previously; and an oxidizing composition (B) comprising:
at least one chemical oxidizing agent vi) as defined previously;
at least one of the compositions (A) and (B) comprising:
   at least iii) one fatty substance as defined previously; and
   optionally at least iv) one metal catalyst as defined previously;

it being understood that the content of fatty substance of the composition according to the invention resulting from the mixing of compositions (A)+(B) is greater than or equal to 10% by weight, relative to the total weight of the composition, preferably greater than or equal to 20%, better still greater than or equal to 30% by weight and even better still greater than or equal to 40% by weight.

According to one variant, the preceding process uses a dye composition which is derived from the mixing of three compositions, the three compositions being aqueous or at least one of them being anhydrous.

In a particular process of the invention, two aqueous compositions (B′) and (C′) and one anhydrous composition (A′) are used,
the anhydrous composition (A′) comprising at least iii) one fatty substance as defined previously,
composition (B′) comprising:
   at least i) one oxidation base as defined previously;
   at least ii) one heteroaryl coupler of formula (I) or (I′) as defined previously; and
composition (C′) comprising:
   at least vi) one chemical oxidizing agent as defined previously; it being understood that:
optionally at least v) one alkaline agent as defined previously; which is included in compositions (A′) and/or (B′) and preferably solely in composition (B′);
optionally at least iv) one metal catalyst as defined previously being present in at least one of the three compositions (A′), (B′) or (C′);
these three compositions being such that the content of fatty substance of the dye composition resulting from the mixing of the three compositions (A′)+(B′)+(C′) is greater than or equal to 10%, in particular greater than or equal to 20%, more particularly greater than or equal to 30% and preferably greater than or equal to 40% by weight relative to the total weight of the composition derived from the mixing of (A′)+(B′)+(C′).

Preferably, composition (A′) has a content of fatty substance of greater than or equal to 20%, in particular greater than or equal to 30%, more particularly greater than or equal to 40% and preferably greater than or equal to 40% by weight relative to the total weight of composition (A′).

The mixture obtained according to the preceding processes is then applied to the keratin fibres. After a leave-on time of approximately 3 to 50 minutes, preferably approximately 5 to 30 minutes, the keratin fibres are rinsed, washed with shampoo, rinsed again and then dried.

According to one embodiment, the processes as described above comprise a step of applying to the keratin fibres a composition comprising at least one metal catalyst, preferably before the composition according to the invention comprising at least compounds i) and ii).

The ready-to-use composition that is finally applied to the keratin fibres may be in various forms, such as in the form of liquids, creams or gels or in any other form that is suitable for dyeing keratin fibres, and especially human hair.

### Kit:

A subject of the invention is also a multi-compartment dyeing device or “kit” comprising a first compartment containing composition (A) as defined previously in the dyeing process, and at least a second compartment containing composition (B) as defined previously in the process, the compositions of the compartments being intended to be mixed before application, the composition after mixing of (A)+(B) is such that the amount of fatty substance is greater than or equal to 10%, in particular greater than or equal to 20%, more particularly greater than or equal to 30% and preferably greater than or equal to 40% by weight relative to the total weight of the composition derived from the mixing of (A)+(B). According to one variant, the multi-compartment device of the invention is such that a first compartment contains a dye composition comprising ingredients i) and ii) and optionally iii) to v) as defined previously and a second compartment contains at least vi) one chemical oxidizing agent as defined previously.

The invention also relates to a second multi-compartment device comprising a first compartment containing composition (A′) as described above and a second compartment containing a cosmetic composition (B′) as described above and at least a third compartment comprising composition (C′) as described above, the compositions of the compartments being intended to be mixed before application to give the composition according to the invention; the amount of fatty substance in the composition is greater than or equal to 10% by weight, in particular greater than or equal to 20% by weight, more particularly greater than or equal to 30% by weight and preferably greater than or equal to 40% by weight relative to the total weight of the composition derived from the mixing of compositions (A′), (B′) and (C′).

These devices or kits may be equipped with a means for dispensing the desired mixture on the hair, such as the devices described in patent FR-2 586 913 in the name of the Applicant.

Using these devices, it is possible to dye the keratin fibres by means of a process that involves mixing a dye composition comprising at least one i) oxidation base and at least ii) one coupler of formula (I) as defined previously and with at least vi) one chemical oxidizing agent, and applying the mixture obtained to the keratin fibres for a time that is sufficient to develop the desired colouring.

The examples that follow serve to illustrate the invention without, however, being limiting in nature.

### EXAMPLES

A dye composition is prepared at the time of use by mixing the three compositions A, B and C below:

3.35 g of composition A

1.35 g of composition B

5 g of composition C.

Composition A

| | | |
|---|---|---|
| Liquid petroleum jelly | 64.5 | g |
| 2-Octyldodecanol | 11.5 | g |
| Distearyldimethylammonium-modified hectorite | 3 | g |
| Propylene carbonate | 1 | g |
| Oxyethylenated (4 OE) sorbitan monolaurate | 11 | g |
| Glycol distearate | 8 | g |
| Oxyethylenated (2 OE) lauryl alcohol | 1 | g |

Composition B

| | | |
|---|---|---|
| Oxidation base* a, b or c | 20 × 10⁻³ | mol % |
| Compound of formula (I)** 1, 2, 3, 4 or 5 | 20 × 10⁻³ | mol % |
| Monoethanolamine | 15.3 | g |
| Sodium metabisulfite | 0.7 | g |
| L-Ascorbic acid | 0.25 | g |
| Propylene glycol | 6.2 | g |
| Ethanol | 15.5 | g |
| Hexylene glycol | 3 | g |
| Dipropylene glycol | 3 | g |
| Benzyl alcohol | 7 | g |
| EDTA | 0.287 | g |
| Water | qs 100 | g |

Oxidation base* 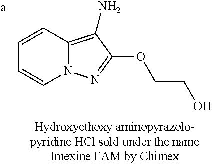 [SMILES: Nc1c(OCCO)nn2ccccc12] 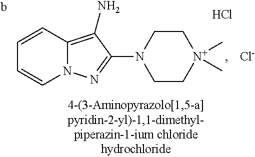 [SMILES: C[N+]1(C)CCN(c2nn3ccccc3c2N)CC1 ; Cl ; [Cl-]] 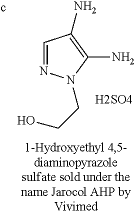
Compound of formula (I)**

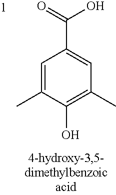

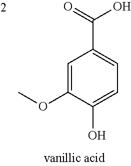

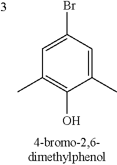

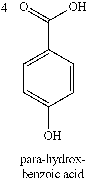

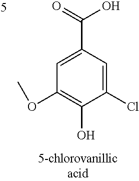

Composition C (Oxidizing Agent)

| | | |
|---|---|---|
| 50% hydrogen peroxide solution | 12 | g |
| Liquid petroleum jelly | 20 | g |
| Cetylstearyl alcohol (30/70 C16/C18) | 8 | g |
| Oxyethylenated cetylstearyl alcohol (33 OE) | 3 | g |
| Tetrasodium pyrophosphate, decahydrate | 0.03 | g |
| Sodium hexahydroxystannate | 0.04 | g |
| Diethylenetriaminepentaacetic acid, pentasodium salt as | 0.15 | g |
| a 40% aqueous solution | | |
| Polydimethyldiallylammonium chloride at 40% in water, | 0.5 | g |
| non-stabilized | | |
| Poly[(dimethyliminio)-1,3-propanediyl(dimethylimino)-1,6- | 0.25 | g |
| hexanediyl dichloride] as an aqueous 60% solution | | |
| Protected oxyethylenated (4 OE) rapeseed acid amides | 1.3 | g |
| Vitamin E | 0.1 | g |
| Glycerol | 0.5 | g |

| | |
|---|---|
| Phosphoric acid | qs pH 2.2 |
| Water | qs 100 |

Each of the compounds of formulae 1, 2, 3, 4 and 5 is applied in combination with each of the heterocyclic bases a, b and c:

Dyeing Process

Locks of natural Caucasian hair containing 90% of grey hairs (NG) are treated with each dye composition obtained from 9.7 g of the mixture A+B+C, which is then left on for 35 minutes at room temperature.

After this leave-on time, the locks are washed with iNOA POST shampoo, rinsed and then dried under a hood.

Colorimetric Results:

The colouring of the hair is evaluated using a Minolta CM-3600D spectrocolorimeter (illuminant D65, angle 10°, SCI values) for the L*, a*, b* colorimetric measurements.

In this L*a*b* system, L* represents the intensity of the colour, a* indicates the green/red colour axis and b* the blue/yellow colour axis. The lower the value of L*, the darker or more intense the colour. The higher the value of a*, the redder the shade, and the higher the value of b*, the yellower the shade.

The variation in colouring between the dyed locks of natural grey hair that are untreated (control) and after treatment are defined by (ΔE*):

ΔE*=\sqrt{(L*−Lₒ*)²+(a*−aₒ*)²+(b*−bₒ*)²}

In this equation, L*, a* and b* represent the values measured on locks of hair after treatment and L*₀, a₀* and b₀* represent the values measured on untreated hair.

The higher the value of ΔE, the greater the difference in colour between the control locks (non-dyed) and the dyed locks, i.e. the greater the colour build-up.

The colorimetric results are collated in the table below.

| | | L* | a* | b* | δE*ab |
|---|---|---|---|---|---|
| | Colour | (D65) | (D65) | (D65) | (D65) |
| Control | — | 60.4 | 0.75 | 13.95 | — |

Compound 1

| | | | | | |
|---|---|---|---|---|---|
| +base a | violet | 19.8 | 16.2 | −5.66 | 47.7 |
| +base b | blue | 18.6 | 2.69 | −15.3 | 51 |
| +base c | red | 25.4 | 32.9 | 3.88 | 48.6 |

Compound 2

| | | | | | |
|---|---|---|---|---|---|
| +base a | violet | 31.5 | 17.6 | −4.59 | 38.2 |
| +base b | green | 47.9 | −4.01 | 4.33 | 16.5 |
| +base c | red | 29.9 | 28.6 | −1.95 | 44.3 |

Compound 3

| | | | | | |
|---|---|---|---|---|---|
| +base a | violet | 21.2 | 17.3 | −3.97 | 46.2 |

Compound 4

| | | | | | |
|---|---|---|---|---|---|
| +base a | nacreous | 33.5 | 12.4 | −1.02 | 32.9 |
| | brown | | | | |

Compound 5

| | | | | | |
|---|---|---|---|---|---|
| +base a | violet | 36.48 | 13.74 | 2.29 | 33.77 |

The locks treated with the compositions according to the invention have high ΔE values, i.e. good colour build-up.

The compositions according to the invention lead to powerful, varied colourings.

1. Composition comprising:
i) at least one coupler of formula (I), and also organic or mineral acid or base salts thereof, optical or geometrical isomers thereof, tautomers thereof or solvates thereof: 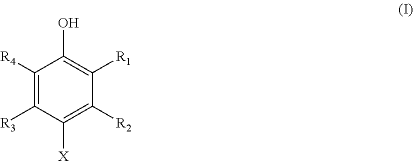
in which formula (I):
X represents a halogen atom, a COOH group or an amido group
—C(O)—NR₅R₆ with R₅ and R₆, which may be identical or different, representing a hydrogen atom or an alkyl group,
R₁ to R₄, which may be identical or different, represent a hydrogen or halogen atom, an alkyl group or an alkoxy group,
and
ii) at least one oxidation base.

2. Composition according to the preceding claim, in which the compound of formula (I) is such that X represents a halogen atom, preferably bromine or chlorine, or a COOH group.

3. Composition according to claim 1 or 2, in which the compound of formula (I) is such that R₁ to R₄, which may be identical or different, represent a hydrogen or halogen atom; a C₁-C₁₀, preferably C₁-C₈ and better still C₁-C₆ alkyl group, or a C₁-C₁₀, preferably C₁-C₈ and better still C₁-C₆ alkoxy group.

4. Composition according to one of the preceding claims, in which the compound of formula (I) is such that R₂ and R₃ represent a hydrogen atom.

5. Composition according to one of the preceding claims, in which the compound of formula (I) is such that R₁ represents a hydrogen atom, a C₁-C₆ alkyl group, preferably a methyl group, or a C₁-C₆ alkoxy group, preferably a methoxy group.

6. Composition according to one of the preceding claims, in which the compound of formula (I) is such that R₄ represents a hydrogen or halogen atom, preferably bromine or chlorine, a C₁-C₆ alkyl group, preferably a methyl group, or a C₁-C₆ alkoxy group, preferably a methoxy group.

7. Composition according to one of the preceding claims, in which the compound of formula (I) is such that :
X represents a COOH group,
R₂ and R₃ represent a hydrogen atom and
R₁ and R4 represent a C₁-C₆ alkyl group, preferably a methyl group.

8. Composition according to one of the preceding claims, in which the compound of formula (I) is chosen from the following compounds: 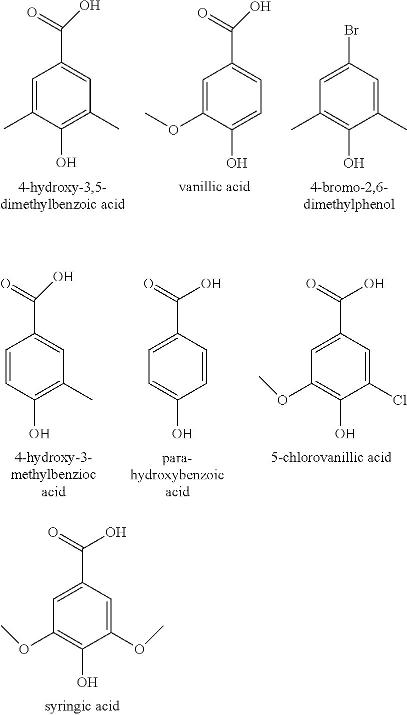
and mixtures thereof.

9. Composition according to one of the preceding claims, in which the compound of formula (I) is 4-hydroxy-3,5-dimethylbenzoic acid.

10. Composition according to any one of the preceding claims, in which the oxidation base(s) are chosen from para-phenylenediamines, bis(phenyl)alkylene diamines, para-aminophenols, ortho-aminophenols and heterocyclic bases, and the addition salts thereof; preferably chosen from heterocyclic bases such as pyridine derivatives, pyrimidine derivatives and pyrazole derivatives.

11. Composition according to any one of the preceding claims, in which the oxidation base(s) are chosen from para-phenylenediamines and bis(phenyl)alkylene diamines and more particularly from N,N-bis-(β-hydroxyéthyl) paraphénylènediamine and salts thereof.

12. Composition according to any one of the preceding claims, in which the oxidation base(s) are chosen from heterocyclic bases, such as pyridine derivatives, pyrimidine derivatives and pyrazole derivatives

13. Composition according to any one of the preceding claims, in which the oxidation base(s) ii) are chosen from the compounds of formula (II) below, addition salts thereof or solvates thereof: 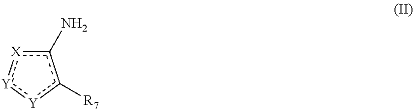
in which formula (II):
X denotes a group —CO— or —CR₁₀—,
Y denotes a nitrogen atom or a group —NR₈,
Y′ denotes a nitrogen atom or a group —NR₉,
with R₁₀ representing a hydrogen atom or a C₁-C₄ alkyl group, optionally substituted with one or more hydroxyl or amino groups,
R₈ representing a C₁-C₄ alkyl group substituted with one or more hydroxyl or amino groups,
R₉ representing a C₁-C₄ alkyl group optionally substituted with one or more hydroxyl or amino groups,
it being possible for R₈ and R₉, on the one hand, and for R₉ and R₁₀, on the other hand, to form, with the atoms bearing them, a saturated or unsaturated, 5- to 7-membered heterocycle, optionally substituted with one or more halogen atoms or one or more hydroxyl, amino or C₁-C₄ alkyl groups,
R₇ representing an amino group, a C₁-C₄ alkyl group optionally substituted with one or more hydroxyl or amino groups, or a C₁-C₄ alkoxy group optionally substituted with one or more hydroxyl or amino groups,
the ring system containing X, Y and Y′ comprising at least one double bond.

14. Composition according to any one of the preceding claims, characterized in that the oxidation base(s) ii) are chosen from the compounds of formula (II′) below, addition salts thereof or solvates thereof: 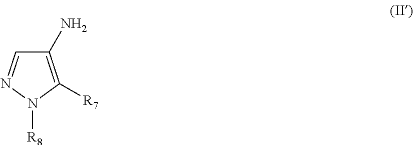
R₇ representing an amino group, a C₁-C₄ alkyl group optionally substituted with one or more hydroxyl or amino groups, or a C₁-C₄ alkoxy group optionally substituted with one or more hydroxyl or amino groups, and
R₈ representing a C₁-C₄ alkyl group substituted with one or more hydroxyl or amino groups.

15. Composition according to claim 14, characterized in that the oxidation bases of formula (II′) are such that R₈ and R₉, on the one hand, or R₉ and R₁₀, on the other hand, form, with the atoms bearing them, a saturated or unsaturated, 5- to 7-membered heterocycle optionally substituted with one or more halogen atoms, or one or more hydroxyl, amino and/or C₁-C₄ alkyl groups.

16. Composition according to any one of the preceding claims, characterized in that the oxidation base(s) ii) are chosen from:
a) the compounds of formula (II″), addition salts thereof and solvates thereof: 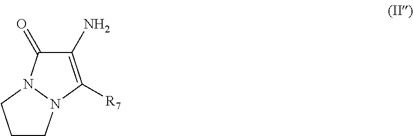
R₇ representing an amino group, a C₁-C₄ alkyl group optionally substituted with one or more hydroxyl or amino groups, or a C₁-C₄ alkoxy group optionally substituted with one or more hydroxyl or amino groups, R₇ preferably representing an amino group;
b) the compounds of formula (II′″), addition salts thereof and solvates thereof: 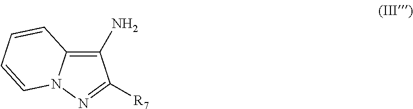
R₇ representing an amino group, a C₁-C₄ alkyl group optionally substituted with one or more hydroxyl or amino groups, or a C₁-C₄ alkoxy group optionally substituted with one or more hydroxyl or amino groups, R₇ preferably representing a C₁-C₄ alkoxy group optionally substituted with one or more hydroxyl groups, in particular a hydroxyethoxy group;
and mixtures thereof.

17. Composition according to any one of the preceding claims, characterized in that the oxidation bases ii) are chosen from 2-(4,5-diamino-1H-pyrazol-1-yl)ethanol, 2,3-diamino-6,7-dihydro-1H,5H-pyrazolo[1,2-A]pyrazol-1-one and 2-[3-aminopyrazolo[1,5-a]pyridin-2-yl]oxy]ethanol, 4-(3-aminopyrazolo[1,5-a]pyridin-2-yl)-1,1-dimethylpiperazin-1-ium or an addition salt thereof, and mixtures thereof.

18. Composition according to any one of the preceding claims, characterized in that it comprises at least one fatty substance, which is preferably liquid, in a content of greater than or equal to 10% by weight, preferably greater than or equal to 20% by weight, better still greater than or equal to 30% by weight, and even better still greater than or equal to 40% by weight, relative to the total weight of the composition.

19. Composition according to the preceding claim, characterized in that the fatty substance(s) are chosen from liquid petroleum jelly, C₆-C₁₆ alkanes, polydecenes, liquid esters of fatty acids and/or of fatty alcohols, and liquid fatty alcohols, or mixtures thereof, preferably from liquid petroleum jelly and octyldodecanol.

20. Composition according to any one of the preceding claims, characterized in that it comprises one or more chemical oxidizing agents, the chemical oxidizing agent preferably being hydrogen peroxide.

21. Process for dyeing keratin fibres, in particular human keratin fibres such as the hair, which consists in using the composition according to any one of claims 1 to 20, in the presence of one or more oxidizing agents.

22. Process according to the preceding claim, in which the dye composition is derived from the mixing of two compositions:
a composition (A) comprising:
at least one coupler i) of formula (I) as defined in any one of claims 1 to 9;
at least one oxidation base ii) as defined in any one of claims 10 to 17;
optionally at least one alkaline agent; and
a composition (B) comprising:
at least one chemical oxidizing agent;
at least one of the compositions (A) and (B) comprising:
at least iii) one fatty substance as defined in claim 19, and
optionally at least one metal catalyst,
it being understood that the content of fatty substance of the composition resulting from the mixing of compositions (A)+(B) is greater than or equal to 10%, in particular greater than or equal to 20%, more particularly greater than or equal to 30% and preferably greater than or equal to 40% by weight relative to the total weight of the composition.

23. Process according to claim 21, in which the composition is derived from the mixing of three compositions:
an anhydrous composition (A′) comprising at least iii) one fatty substance as defined in claim 19;
a composition (B′) comprising:
at least i) one coupler of formula (I) as defined in any one of claims 1 to 9;
at least ii) one oxidation base as defined in any one of claims 10 to 17; and
a composition (C′) comprising at least one chemical oxidizing agent;
it being understood that:
at least one alkaline agent is included in compositions (A′) and/or (B′) and preferably solely in composition (B′);
optionally at least one metal catalyst is included in at least one of the compositions (A′), (B′) or (C′);
these three compositions being such that the content of fatty substance of the dye composition resulting from the mixing of the three compositions (A′)+(B′)+(C′) is greater than or equal to 10%, in particular greater than or equal to 20%, more particularly greater than or equal to 30% and preferably greater than or equal to 40% by weight relative to the total weight of the composition derived from the mixing of (A′)+(B′)+(C′).

24. Process according to any one of claims 21 to 23, characterized in that it comprises a step of applying to the keratin fibres a composition comprising at least one metal catalyst, preferably before the dye composition according to any one of claims 1 to 20.
